(19) United States
(12) Patent Application Publication
(10) Pub. No.: US 2024/0376328 A1
(43) Pub. Date: Nov. 14, 2024

(54) PHOTOCURABLE COMPOSITION

(71) Applicant: CANON KABUSHIKI KAISHA, Tokyo (JP)

(72) Inventors: Fen WAN, Austin, TX (US); Weijun LIU, Cedar Park, TX (US)

(21) Appl. No.: 18/315,249

(22) Filed: May 10, 2023

(51) Int. Cl. C09D 11/101; C07C 69/604; C07D 239/04; C07D 249/16

(52) U.S. Cl. CPC C09D 11/101; C07C 69/604; C07D 239/04; C07D 249/16

(57) ABSTRACT

A photocurable composition can comprising a polymerizable material, a hindered stabilizer, and a photoinitiator, wherein the polymerizable material comprises at least one multi-functional aromatic vinyl monomer; and the hindered stabilizer is a hindered amine of formula (1) or a hindered phenol of formula (2)

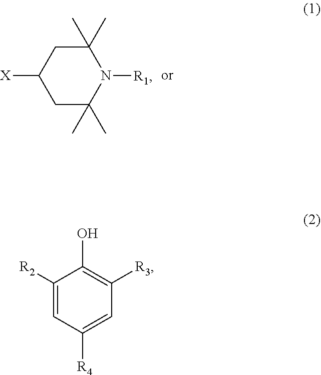

wherein X is H, CH₃, or Y—Z; Y is CH₂, O, S, or N; Z is an organic substituent; R₁ is H, CH₃, OH, OR₅, CO—CH₃, or C(═O)R₅; R₂, R₃, R₄, R₅ is an organic substituent.

### FIELD OF THE DISCLOSURE

The present disclosure relates to a photocurable composition, particularly to a photocurable composition for inkjet adaptive planarization adapted for forming photo-cured layers.

### BACKGROUND

Inkjet Adaptive Planarization (IAP) is a process which planarizes a surface of a substrate, e.g., a wafer containing an electric circuit, by jetting liquid drops of a photocurable composition on the surface of the substrate and bringing a flat superstrate in direct contact with the added liquid to form a flat liquid layer. The flat liquid layer is typically solidified under UV light exposure, and after removal of the superstrate a planar polymeric surface is obtained, which can be subjected to subsequent processing steps, for example, baking, etching, and/or further deposition steps.

Subsequent baking of the formed photo-cured layer is often conducted at temperatures above its glass transition temperature and close to its melting point, and requires a high thermal stability and low shrinkage.

There exists a need for improved IAP materials, wherein the photocurable compositions have a long shelf life and which can form planar photo-cured layers with high thermal stability during subsequent processing.

### SUMMARY

In one embodiment, a photocurable composition can comprise a photocurable composition comprising a polymerizable material, a hindered stabilizer, and a photoinitiator, wherein the polymerizable material comprises at least one multi-functional aromatic vinyl monomer; and the hindered stabilizer may be a hindered amine of formula (1) or a hindered phenol of formula (2)

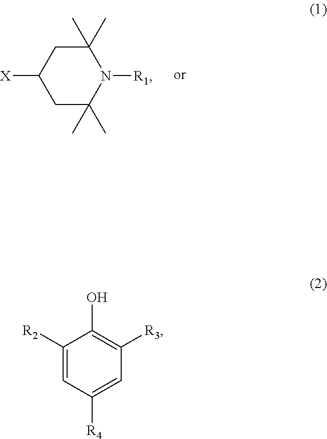

wherein X is H, CH₃, or Y—Z; Y is CH₂, O, S, or N; Z is an organic substituent; R₁ is H, CH₃, OH, OR₅, CO—CH₃, or C(═O)R₅; R₂, R₃, R₄, R₅ is an organic substituent.

In one aspect of the photocurable composition, the hindered stabilizer can have a structure of formula (1), wherein X is Y—Z, with Z comprising at least one reactive C═C group.

In certain aspects of the photocurable composition, the hindered stabilizer can be a hindered amine selected from the group of:

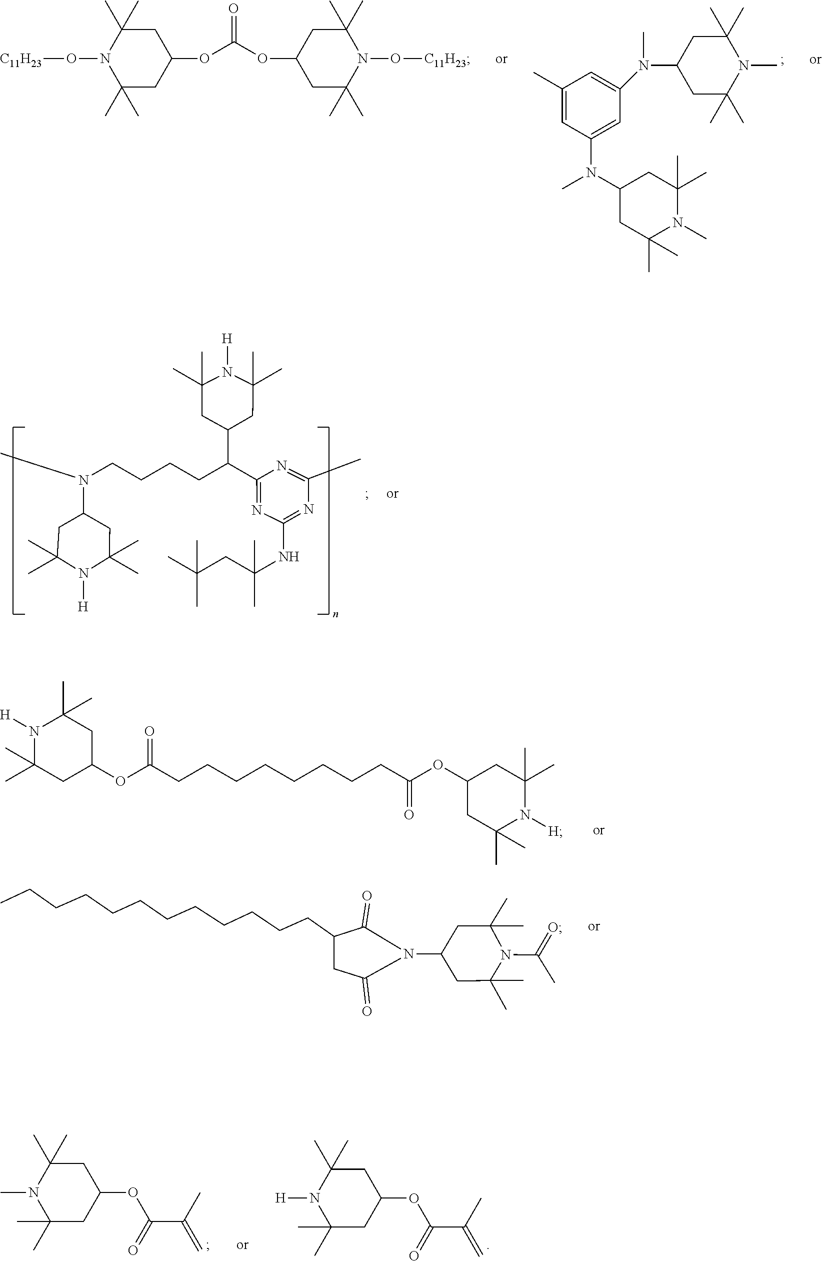

In another aspect, the hindered stabilizer can have a molecular weight of at least 600 g/mol.

In further aspects of the photocurable composition, the hindered stabilizer can be a hindered phenol selected from the group of:

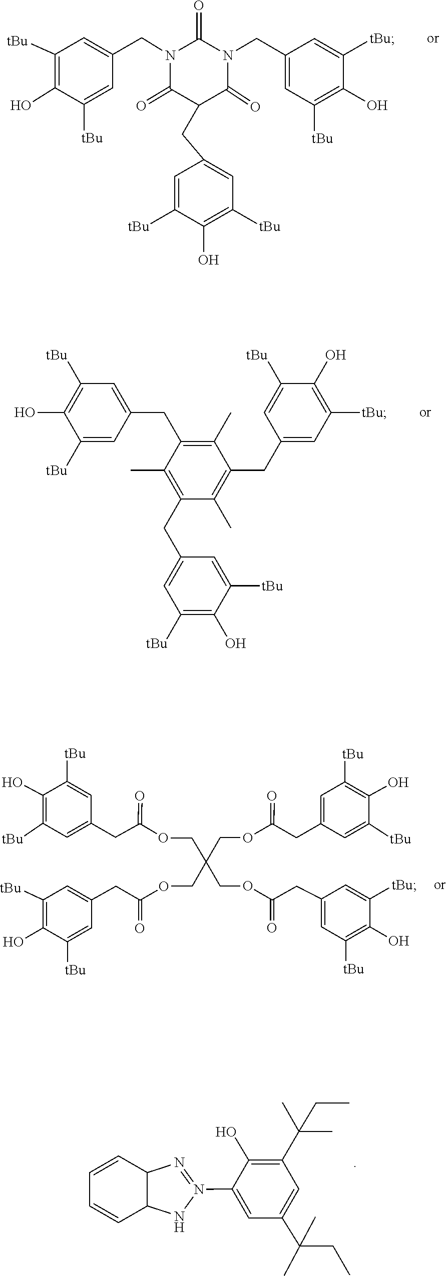

In one embodiment of the photocurable composition, the amount of the hindered stabilizer can be at least 0.5 wt % and not greater than 5 wt %.

In another embodiment, the at least one multi-functional aromatic vinyl monomer of the polymerizable material can include a divinylbiphenyl monomer (DVBPh), or a trivinylbiphenyl monomer (TVBPh), or a trivinylphenyl monomer (TVPh), or a combination thereof.

In one aspect, the at least one multi-functional aromatic vinyl monomer can comprise at least one vinyl group and at least one acrylate group.

In certain aspects, the multi-functional aromatic vinyl monomer of the polymerizable material can be selected from:

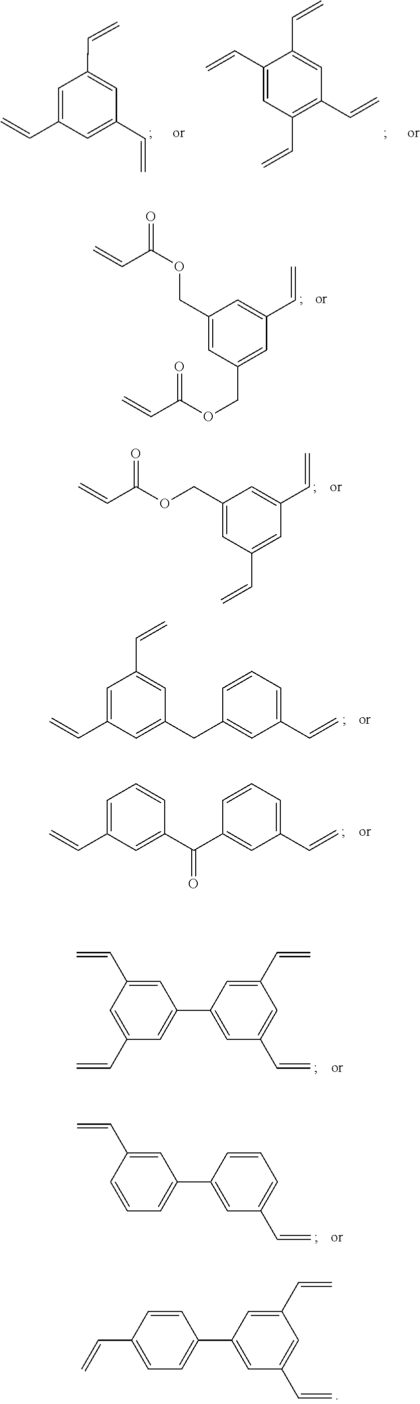

In one aspect of the photocurable composition, the amount of the multi-functional vinyl monomer can be at least 80 wt % based on the total weight of the polymerizable material. In another aspect, the amount of the multi-functional vinyl monomer may be at least 95 wt % based on the total weight of the polymerizable material.

In a further embodiment of the photocurable composition, the amount of the polymerizable material can be at least 90 wt % based on the total weight of the photocurable composition.

In one aspect, the carbon content of the photocurable composition after photo-curing can be at least 71 percent.

In another aspect, the viscosity of the photocurable composition may be not greater than 30 mPa·s.

In a certain aspect, the photocurable composition can be essentially free of a solvent.

In one embodiment, a laminate can comprise a substrate and a photo-cured layer overlying the substrate, wherein the photo-cured layer is formed from the photocurable composition of the present disclosure.

In one aspect of the laminate, the initial degradation temperature T(X) of the photo-cured layer can be at least 330° C.

In another embodiment, a method of forming a photo-cured layer on a substrate can comprise: applying a layer of a photocurable composition on the substrate, wherein the photocurable composition can comprise a polymerizable material, a hindered stabilizer, and a photoinitiator, wherein the polymerizable material comprises at least one multi-functional aromatic vinyl monomer; and the hindered stabilizer is a hindered amine of formula (1) or a hindered phenol of formula (2):

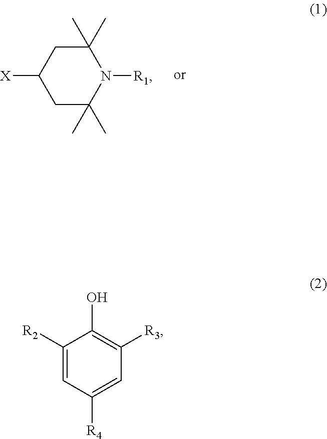

with X being H, CH₃, or Y—Z, wherein Y is CH₂, O, or S, or N, and Z is an organic substituent; R₁ is H, CH₃, OH, OR₅, or CO—CH₃ or COR₅; R₂, R₃, R₄, R₅ are organic substituents, the same or different; bringing the photocurable composition into contact with a superstrate or an imprint template; irradiating the photocurable composition with light to form a photo-cured layer; and removing the superstrate or the imprint template from the photo-cured layer.

In one aspect of the method, the initial degradation temperature T(X) of the photo-cured layer can be at least 330° C.

In a further embodiment, a method of manufacturing an article can comprising: forming a photo-cured layer on a substrate as described above, forming a pattern on the substrate; and processing the substrate on which the pattern has been formed in the forming; and manufacturing the article from the substrate processed in the processing.

### BRIEF DESCRIPTION OF THE DRAWINGS

Embodiments are illustrated by way of example and are not limited in the accompanying figures.

FIG. 1 includes a graph illustrating TGA curves obtained via dynamic thermogravimetry of photo-cured materials made from photocurable compositions of Example 1 according to embodiments.

FIG. 2 includes a graph illustrating TGA curves obtained via isothermal thermogravimetry at 350° C. of photo-cured materials made from photocurable compositions of Example 1 according to embodiments.

FIG. 3 includes a graph illustrating TGA curves obtained via dynamic thermogravimetry of photo-cured materials made from photocurable compositions of Example 2 according to embodiments.

FIG. 4 includes a graph illustrating TGA curves obtained via isothermal thermogravimetry at 400° C. of photo-cured materials made from photocurable compositions of Example 2 according to embodiments.

### DETAILED DESCRIPTION

The following description is provided to assist in understanding the teachings disclosed herein and will focus on specific implementations and embodiments of the teachings. This focus is provided to assist in describing the teachings and should not be interpreted as a limitation on the scope or applicability of the teachings.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and may be found in textbooks and other sources within the imprint and lithography arts.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus.

As used herein, and unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

The present disclosure is directed to a photocurable composition comprising a polymerizable material, a hindered stabilizer, and a photoinitiator, wherein the polymerizable material can comprise at least one multi-functional aromatic vinyl monomer, and the hindered stabilizer can be a hindered amine of formula (1) or a hindered phenol of formula (2):

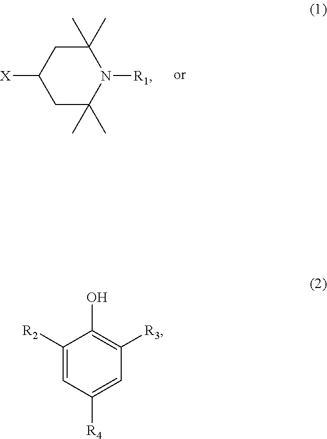

wherein X is H, CH₃, or Y—Z; Y is CH₂, O, S, or N; Z is an organic substituent;
R₁ is H, CH₃, OH, OR₅, CO—CH₃, or C(═O)R₅; R₂, R₃, R₄, R₅ are organic substituents, wherein each organic substituent can be different, or the same, or at least partially the same substituent.

Not being bound to theory, a photocurable composition comprising certain combinations of aromatic vinyl monomers and hindered stabilizer having the structure of formula (1) or formula (2) can have advantages of having a long shelf life and of forming photo-cured layers during IAP processing with high thermal stability and high etch resistance.

In one embodiment, the hindered stabilizer can be a hindered amine having the structure of formula (1), wherein X is Y—Z, with Z comprising at least one reactive C═C group.

In a particular embodiment, a molecular weight of the hindered stabilizer can be at least 230 g/mol, or at least 250 g/mol, or at least 300 g/mol, or at least 400 g/mol, or at least 440 g/mol, or at least 500 g/mol, or at least 550 g/mol, or at least 600 g/mol. In another aspect, the molecular weight of the hindered stabilizer may be not greater than 3000 g/mol, or not greater than 2500 g/mol, or not greater than 2000 g/mol, or not greater than 1000 g/mol, or not greater than 800 g/mol, or not greater than 500 g/mol.

Non-limiting examples of the hindered amine stabilizers can be one or more stabilizers of structures (3) to (9):

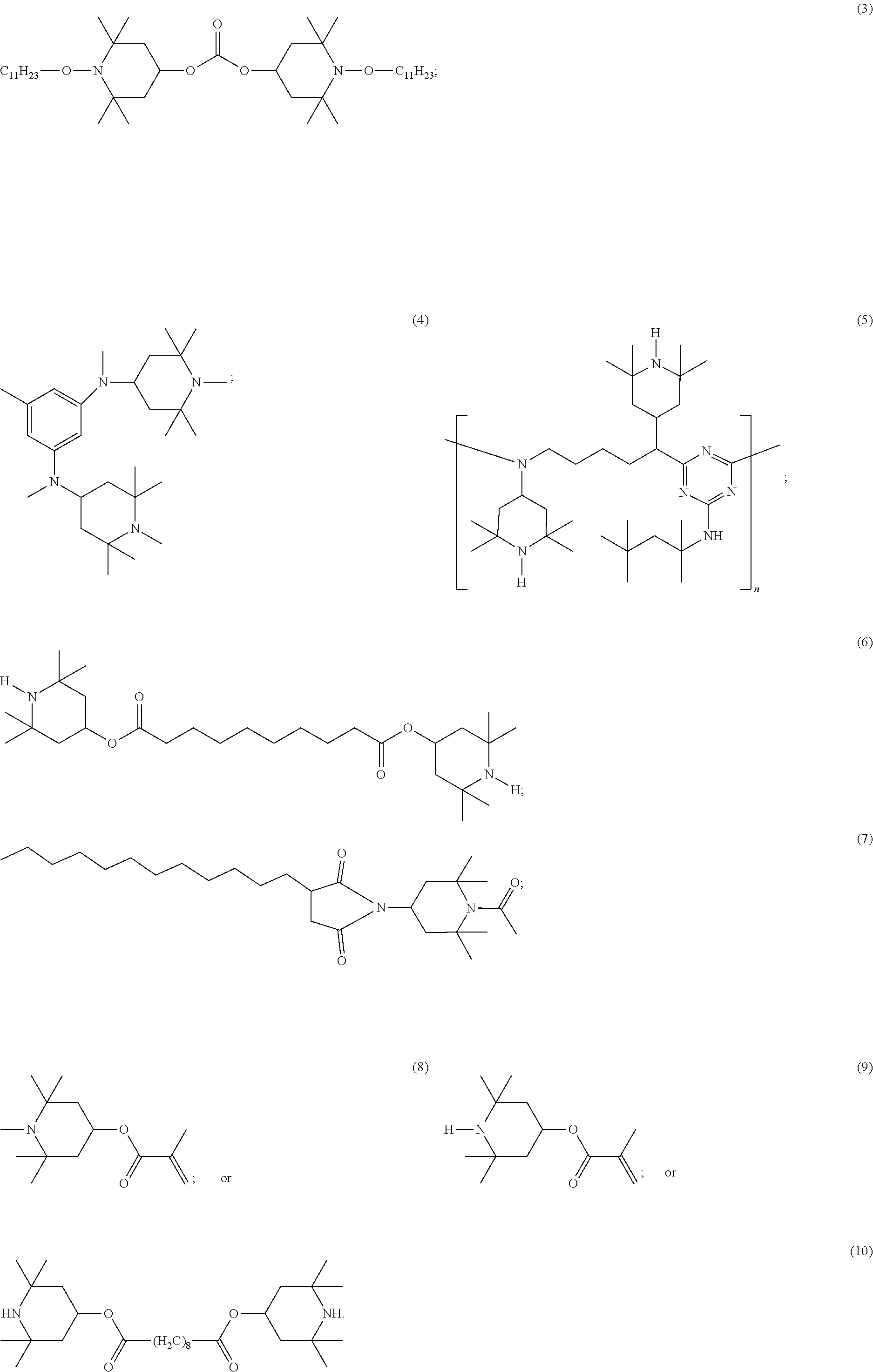

In another embodiment, the hindered stabilizer can be a hindered phenol having the structure of formula (2). Non-limiting examples of hindered phenol stabilizers falling under the structure of formula (2) can be the following structures (11) to (14).

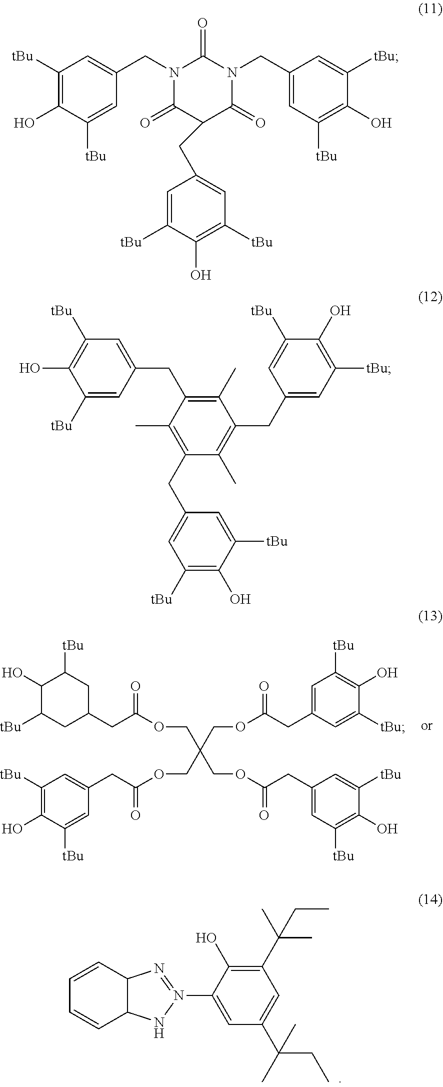

In a certain aspect, the hindered stabilizer can be also a combination of at least one hindered amine of formula (1) and at least one hindered phenol of formula (2).

In one embodiment, the amount of the hindered stabilizer of the photocurable composition of the present disclosure can be at least 0.1 wt % based on the total weight of the photocurable composition, or at least 0.3 wt %, or at least 0.5 wt %, or at least 1 wt %, or at least 2 wt %, or at least 3 wt %, or at least 4 wt %, or at least 5 wt %. In another embodiment, the amount of the hindered stabilizer may be not greater than 10 wt %, or not greater than 8 wt %, or not greater than 5 wt %, or not greater than 3 wt %. In a certain aspect, the amount of the hindered stabilizer can be at least 0.5 wt % and not greater than 5 wt % based on the total weight of the photocurable composition, or at least 1.0 wt % and not greater than 3 wt %.

The polymerizable material of the photocurable composition of the present disclosure can comprise at least one multi-functional aromatic vinyl monomer. In one aspect, the multi-functional aromatic vinyl monomer can comprise at least one aromatic ring, and at least two vinyl groups. In another aspect, the at least one multi-functional aromatic vinyl monomer may comprise at least one aromatic ring, at least one vinyl group, and at least one acrylate group.

In certain aspects, the at least one multi-functional aromatic vinyl monomer can include a divinylbiphenyl monomer (DVBPh), or a trivinylbiphenyl monomer (TVBPh), or a trivinylphenyl monomer (TVPh), or a combination thereof.

Non-limiting examples of multi-functional aromatic vinyl monomers can be one or more of the following monomers:

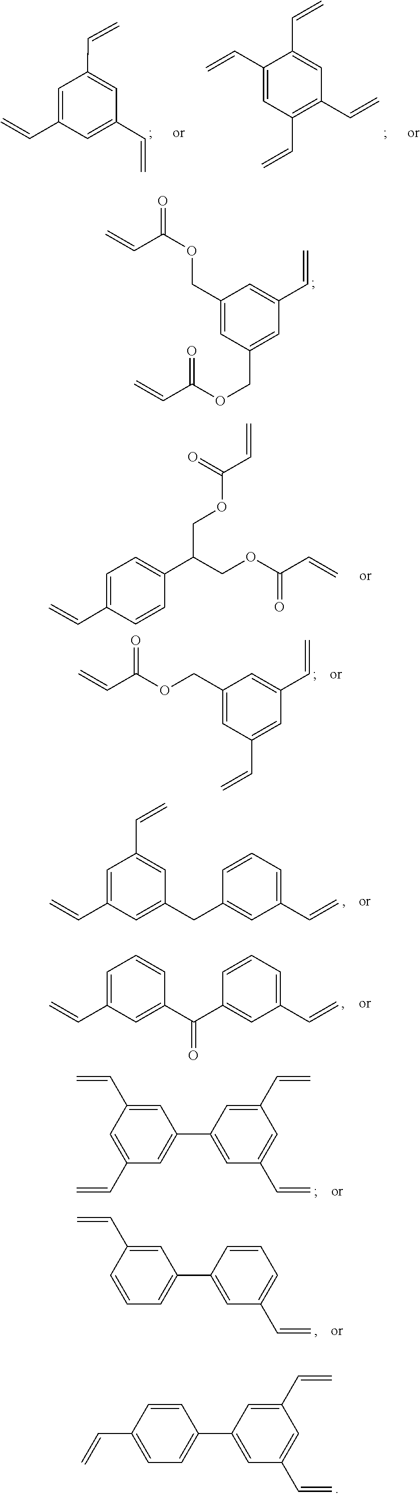

The amount of the multi-functional aromatic vinyl monomer can be at least at least 70 wt % based on the total weight of the polymerizable material, such as at least 75 wt %, at least 80 wt %, at least 85 wt %, at least 90 wt %, or at least 95 wt %. In another aspect, all of the polymerizable material can be one or more of a multi-functional aromatic vinyl monomers (100 wt %), or not greater than 98 wt % based on the total weight of the polymerizable material, or not greater than 95 wt %, or not greater than 90 wt %. In a certain aspect, the amount of the multi-functional aromatic vinyl monomer can range from at least 80 wt % to 100 wt % based on the total weight of the polymerizable material.

In a further aspect, the polymerizable material of the photocurable composition of the present disclosure can further include at least one polymerizable monomer not comprising an aromatic ring, or a polymerizable monomer containing no vinyl group but other functional groups, e.g., one or more acrylate groups. As used herein, the term “acrylate monomer” relates to an unsubstituted or an alkyl-substituted acrylate monomer, for example, a methacrylate monomer.

The amount of polymerizable material in the photocurable composition can be at least 50 wt % based on the total weight of the photocurable composition, such as at least 60 wt %, at least 70 wt %, at least 80 wt %, at least 85 wt %, at least 90 wt %, or at least 95 wt %. In another aspect, the amount of polymerizable material may be not greater than 99 wt %, such as not greater than 98 wt %, or not greater than 97 wt %, or not greater than 95 wt %, or not greater than 90 wt % based on the total weight of the photocurable composition. The amount of polymerizable material can be a value between any of the minimum and maximum values noted above. In a particular aspect, the amount of the polymerizable material can be at least 80 wt % and not greater than 97 wt %.

In another embodiment, the polymerizable material of the photocurable composition can include a certain amount of polymerizable oligomers, or polymers.

The photocurable composition of the present disclosure can be adapted for use in inkjet adaptive planarization (IAP) or in nanoimprint lithography (NIL).

IAP and NIL processing typically employ photocurable compositions with low viscosity. In one aspect, the viscosity of the photo-curable composition of the present disclosure can be not greater than 50 mPa·s, such as not greater than 40 mPa·s, not greater than 30 mPa·s, not greater than 25 mPa·s, not greater than 20 mPa·s. In other certain embodiments, the viscosity may be at least 2 mPa·s, or at least 3 mPa·s, or at least 5 mPa·s. As used herein, all viscosity values relate to viscosities measured at a temperature of 23° C. with the Brookfield method using a Brookfield Viscometer.

The selection of the polymerizable monomer can be made with the aim of making photocurable compositions that may form photo-cured layers with a high carbon content. In one aspect, the carbon content of a formed layer after photo-curing can be at least 71 wt % based on the total weight of the photo-cured layer, or at least 72 wt %, or at least 73 wt %.

In another aspect, the photo-cured layer of the laminate can have an Ohnishi number of not greater than 3.2, or not greater than 3.1, or not greater than 3.0, or not greater than 2.8, or not greater than 2.7, or not greater than 2.6. In another aspect, the Ohnishi number may be at least 1.8, such as at least 1.9, at least 2.0, at least 2.1, at least 2.2, or at least 2.3.

In a particular embodiment, the photo-cured layer can have a carbon content of at least 71% and an Ohnishi number of not greater than 3.1.

The photocurable composition can be adapted that a photo-cured layer formed from the photocurable composition may have a high thermal stability. In one aspect, the onset temperature for the thermal degradation of the of the photo-cured layer may be at least 300° C., or at least 330° C., or at least 350° C., or at least 375° C., or at least 400° C. As used herein, the onset temperature for the thermal degradation is also called “initial degradation temperature T(X),” and relates to the temperature in the TGA curve wherein a deflection of the curve from the almost linear plateau is first observed, shortly before the steep degradation decline of the sample.

In one embodiment, the first photocurable composition of the present disclosure can be essentially free of a solvent.

As used herein, if not indicated otherwise, the term solvent relates to a compound which can dissolve or disperse the polymerizable monomers and hindered stabilizer but does not itself polymerize during the photo-curing of the photocurable composition. The term “essentially free of a solvent” means herein an amount of solvent being not greater than 5 wt % based on the total weight of the photocurable composition. In a certain particular aspect, the amount of the solvent can be not greater than 3 wt %, not greater than 2 wt %, not greater than 1 wt %, or the photocurable composition can be free of a solvent, except for unavoidable impurities.

In order to initiate the photo-curing of the composition if exposed to light, one or more photoinitiators can be included in the photocurable composition.

In a certain aspect, the curing can be also conducted by a combination of light and heat curing.

The photocurable composition can further contain one or more optional additives. Non-limiting examples of optional additives can be stabilizers, dispersants, solvents, surfactants, inhibitors, or any combination thereof.

In one embodiment, the photocurable composition can be applied on a substrate to form a photo-cured layer. As used herein, the combination of substrate and photo-cured layer overlying the substrate is called a laminate.

The present disclosure is further directed to a method of forming a photo-cured layer. The method can comprise applying a layer of the photocurable composition described above over a substrate, bringing the photocurable composition into contact with a template or superstrate; irradiating the photocurable composition with light to form a photo-cured layer; and removing the template or the superstrate from the photo-cured layer.

The substrate and the solidified layer may be subjected to additional processing, for example, an etching process, to transfer an image into the substrate that corresponds to the pattern in one or both of the solidified layers and/or patterned layers that are underneath the solidified layer. The substrate can be further subjected to known steps and processes for device (article) fabrication, including, for example, curing, oxidation, layer formation, deposition, doping, planarization, etching, formable material removal, dicing, bonding, and packaging, and the like.

The photo-cured layer may be further used as an interlayer insulating film of a semiconductor device, such as LSI, system LSI, DRAM, SDRAM, RDRAM, or D-RDRAM, or as a resist film used in a semiconductor manufacturing process.

As further demonstrated in the examples, it has been surprisingly discovered that photocurable compositions containing certain combinations of polymerizable monomers with hindered stabilizers of formula (1) or formula (2) can have a desired property profile especially suitable for IAP and NIL processing.

### EXAMPLES

The following non-limiting examples illustrate the concepts as described herein.

In the following examples, a variety of photocurable compositions were made and tested comprising different hindered stabilizers. Table 1 contains a summary of the hindered stabilizers used in Examples 1-4:

TABLE 1

| | | Molecular | |
|---|---|---|---|
| Name | Class | Weight | Structure |
| HP1 | Hindered phenol | 784 | 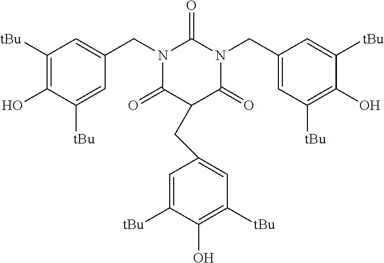 [SMILES: CC(C)(C)c1cc(CC2C(=O)N(Cc3cc(C(C)(C)C)c(O)c(C(C)(C)C)c3)C(=O)N(Cc3cc(C(C)(C)C)c(O)c(C(C)(C)C)c3)C2=O)cc(C(C)(C)C)c1O] |
| HP2 | Hindered phenol | 1178 | 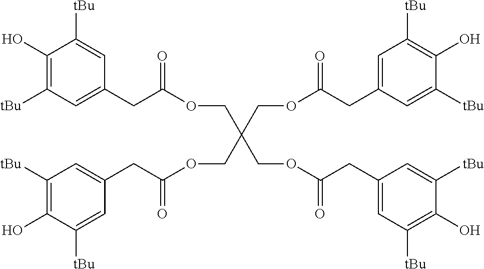 [SMILES: CC(C)(C)c1cc(CC(=O)OCC(COC(=O)Cc2cc(C(C)(C)C)c(O)c(C(C)(C)C)c2)(COC(=O)Cc2cc(C(C)(C)C)c(O)c(C(C)(C)C)c2)COC(=O)Cc2cc(C(C)(C)C)c(O)c(C(C)(C)C)c2)cc(C(C)(C)C)c1O] |
| HP3 | Hindered phenol | 775 | 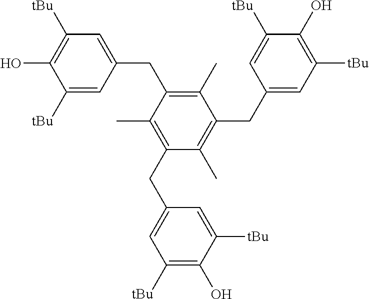 [SMILES: Cc1c(Cc2cc(C(C)(C)C)c(O)c(C(C)(C)C)c2)c(C)c(Cc2cc(C(C)(C)C)c(O)c(C(C)(C)C)c2)c(C)c1Cc1cc(C(C)(C)C)c(O)c(C(C)(C)C)c1] |
| HP4 | Hindered phenol | 351 | 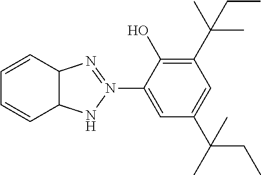 |
| HA1 | Hindered amine | 239 | 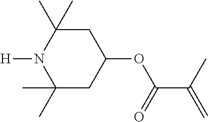 [SMILES: C=C(C)C(=O)OC1CC(C)(C)NC(C)(C)C1] |
| HA2 | Hindered amine | 481 | 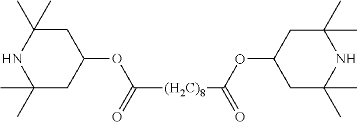 |

### Example 1

Photocurable compositions were prepared by combining 100 parts of polymerizable monomer 5-ethenyl-1,3-benylacrylate (VMXDA), 3 parts photoinitiator Irgacure 819, 1 part surfactant FS3100, and different types of hindered stabilizers in varying amounts. The following hindered stabilizers were used: hindered phenol HP1, hindered phenol HP2, hindered phenol HP3, hindered phenol HP4, and hindered amine HA1. Comparative photocurable composition C1 contained all ingredients as in compositions S1-S8, except a hindered stabilizer.

The different types and amounts of the hindered stabilizers used in the photocurable compositions are summarized in Table 2, together with the viscosities of the photocurable compositions and T(X) of the layers after photo-curing.

TABLE 2

| | | | Amount of | | |
|---|---|---|---|---|---|
| | | | hindered | | Initial |
| | | Hindered | Stabilizer | Viscosity | Degradation |
| Sample | Monomer | Stabilizer | [wt %] | [mPa s] | Temp. T(X) |
| S1 | VMXDA | HP1 | 1 | <23.5* | 328 |
| S2 | VMXDA | HP2 | 1 | <23.5* | 330 |
| S3 | VMXDA | HP3 | 1 | <23.5* | 341 |
| S4 | VMXDA | HP4 | 1 | 22.9 | 324 |
| S5 | VMXDA | HP4 | 2 | 23.7 | |
| S6 | VMXDA | HP4 | 3 | 24.75 | 341 |
| S7 | VMXDA | HA1 | 1 | 22.3 | 338 |
| S8 | VMXDA | HA1 | 3 | 22.47 | 347 |
| C1 | VMXDA | — | — | 22.19 | 320 |

*estimated value

Photo-cured layers were prepared from the photocurable compositions by filling the respective composition in the space between two glass slides, wherein the distance between the two glass slides was 300 microns. Thereafter, the photocurable composition was photo-cured by applying a radiation energy of 5 J.

### Dynamic Thermogravimetry

The thermal stability of the photo-cured layers was investigated via dynamic thermal gravimetric analysis (TGA) using a LINSEIS STA PT1000 instrument (Linseis Messgeraete GmbH, Germany). All measurements were conducted under nitrogen at a rate of 5 liter per hour.

For the TGA measurements, 25-35 mg of the photo-cured sample was placed in a crucible and the initial weight recorded. A reference crucible was used to monitor the weight change of the crucible due to the variation of the temperature. The sample was heated at a rate of 20° C./min and the weight loss of the sample with increasing temperature was recorded at intervals of 1 second. The relative weight percent change was calculated by using the weight loss divided by the total original weight of the sample.

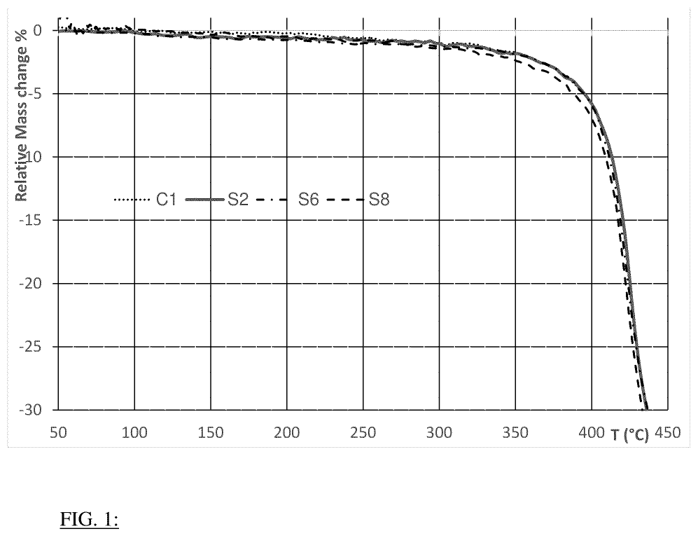

The TGA curves of samples S1-S8 and comparative sample C1 are very similar. FIG. 1 exemplifies the TGA curves of samples S2, S6, S8, and C1. It can be seen that the first phase until the degradation temperature had only a very minor nearly linear weight loss, and the second phase, beginning at the thermal degradation temperature is very steep, until complete degradation of the sample (the curves are only shown up to 30% degradation). The initial degradation temperature T(X) for each curve was identified by determining the point of deflection from an established baseline of phase 1 of the TGA curve and is listed in Table 2.

### Isothermal Thermogravimetry

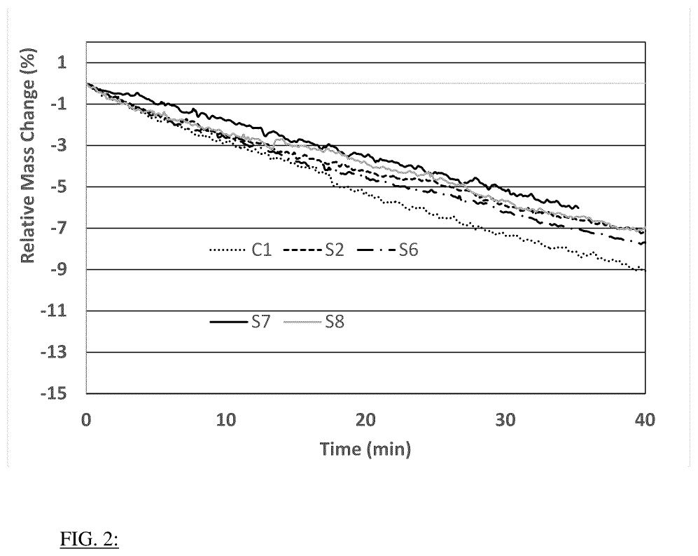

Furthermore, isothermal thermogravimetry (TGA) was conducted at a constant temperature of 350° C. for at least 30 minutes. The measurements were conducted with the same TGA instrument described above and also under nitrogen. For each measurement, 25-35 mg of the photo-cured sample was put in the crucible and the initial weight was recorded. The temperature was quickly raised to 350° C. (within 20 min), and when the temperature of 350° C. was reached, the weight loss was recorded over the time period of 30 minutes. FIG. 2 shows the relative weight loss of photo-cured samples S2, S6, S7, S8, and C1 at a constant temperature of 350° C. over the time period of 30 minutes. It can be seen that Sample C1 had a much faster weight loss than samples S2, S6, S7, and S8.

The speed of the weight loss was characterized by converting the recorded data curve for each sample into a straight line, and expressing the slope of the line in form of a K value (Δweight−loss/minute), wherein the negative slope is expressed as positive number. The K value can be an indication of the thermal stability of the samples, the higher the K-value, the less stable the sample. As shown in Table 3, comparative sample C1 had the highest K value, while samples S2, S6, S7, and S8 had lower K-values. By normalizing the K-values such that the comparative sample is set to 1, it can be seen that sample S8 had an about 28 percent better thermal stability than the comparative sample C1.

TABLE 3

| Sample | K-value | Normalized K-value |
|---|---|---|
| C1 | 0.2405 | 1 |
| S2 | 0.2285 | 0.950 |
| S6 | 0.2127 | 0.884 |
| S7 | 0.1776 | 0.738 |
| S8 | 0.1728 | 0.718 |

### Thermal Shrinkage

The thermal shrinkage was measured by exposing the photo-cured layers to a baking treatment on a 350° C. hot-plate for 2 minutes.

The photo-cured layers for the thermal shrinkage testing were prepared by applying a layer of the liquid photocurable composition on a blank fused silica wafer and photo-curing the liquid layer with light radiation having a radiation wavelength of 365 nm with a total radiation dosage of 5 J. The test was designed that the obtained photo-cured film (before the baking treatment) had a thickness of about 500 nm. The baking treatment of the cured film was conducted by placing it on a hot plate having a temperature of 350° C. for 2 minutes. For calculating the thermal shrinkage, the thickness of the film before and after the baking treatment was measured using a JA Woolam Spectroscopic Ellipsometer M-2000 X-210, and the linear shrinkage (S_{b350}) was calculated according to the following equation: S_{b350}=[(Tₚ−T_{c})/Tₚ]×100%, with Tₚ being the thickness of the liquid film of the photocurable composition before the baking treatment, and T_{c} being the thickness of the photo-cured film after the baking.

The results of the thermal shrinkage measurements are summarized in Table 4. It can be seen that the highest shrinkage occurred with comparative sample C1 (2.6%), while the lowest shrinkage was obtained for sample S4 with 1.55%, which was made by the inclusion of 1 wt % of hindered phenol stabilizer HP4 (see Table 2), which corresponds to a reduction of the shrinkage by about 30 percent.

TABLE 4

| Sample | Hindered stabilizer | Shrinkage [%] |
|---|---|---|
| S4 | 1 wt % HA1 | 1.6 |
| S8 | 3 wt % HP4 | 2.3 |
| C1 | — | 2.6 |

### Example 2

Photocurable compositions were prepared containing as polymerizable monomer 100 parts 3,3′divinylbiphenyl (DVBPH), photo-initiators OXE02 and Omnirad 1316 (each photoinitiator 3 parts), and one part of surfactant FS3100. The photocurable compositions differed by using as hindered stabilizer hindered phenol HP4 (Sample S9), and hindered amine HA2 (sample S10). Comparative sample C2 did not include a hindered stabilizer. A summary of the curable compositions is shown in Table 5.

TABLE 5

| | | | Amount of | | |
|---|---|---|---|---|---|
| | Polym- | | hindered | | Initial |
| | erizable | Hindered | Stabilizer | Viscosity | Degradation |
| Sample | Monomer | Stabilizer | [wt %] | [mPa s] | Temp. T(X) |
| S9 | DVBPH | HP4 | 1 | 16.64 | 368 |
| S10 | DVBPH | HA2 | 1 | 16.23 | 388 |
| C2 | DVBPH | — | | 16.11 | 362 |

Photo-cured layers were prepared from the curable compositions and analyzed via dynamic TGA the same way as described in Example 1.

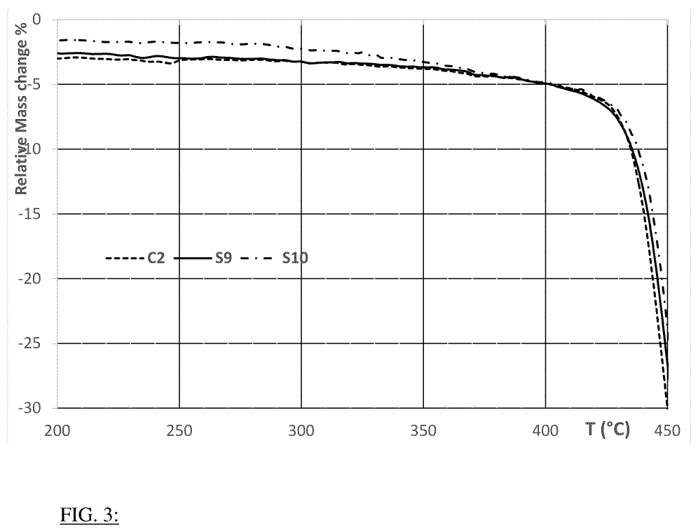

The TGA curves are shown in FIG. 3. It can be seen that sample S10, containing 1 wt % of hindered amine HA2 had a higher initial thermal degradation temperature T(X) than comparative sample C2. The increase of T(X) was about 26° C.

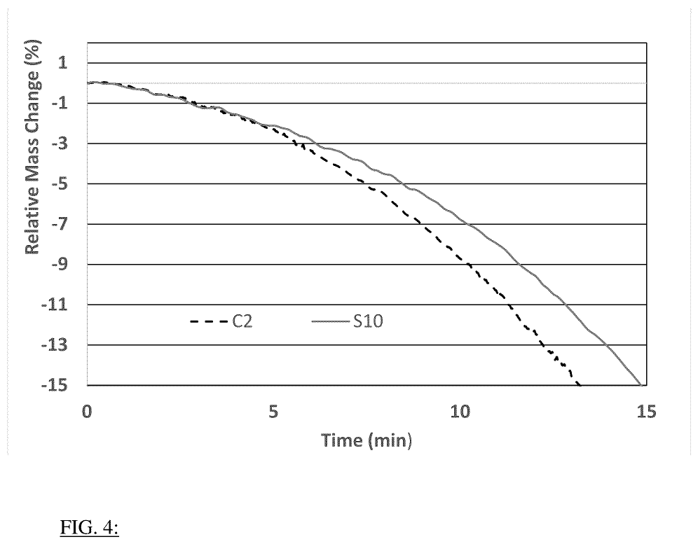

An isothermal TGA was conducted for photo-cured sample S10 and comparative sample C2 at 400° C. As illustrated in FIG. 4, it can be clearly seen that the addition of 1 wt % of hindered amine stabilizer HA2 enhanced the thermal stability and slowed down the degradation.

### Example 3

A third set of photocurable compositions was prepared using as polymerizable monomers 45 parts 3,5-divinylbenzyl acrylate (DVBA); 30 parts 5-ethenyl-1,3-benylacrylate (VMXDA); and 25 parts 3,4′,5-trivinyl-1,1′-biphenyl (3VPH). All curable compositions further contained 1 part FS3100, 3 parts Irgacure 819 and 3 parts OXE02. It was varied the type of hindered stabilizer, being 3 wt % hindered amine HA1 for sample S11, and 3 wt % hindered phenol HP4 for sample S12. Comparative sample C3 did not include a hindered stabilizer but all the other ingredients as in samples S11 and S12. A summary of the photocurable compositions is shown in Table 6.

TABLE 6

| | | | Amount of | | |
|---|---|---|---|---|---|
| | Polym- | | hindered | | Initial |
| | erizable | Hindered | Stabilizer | | Degradation |
| Sample | Monomer | Stabilizer | [wt %] | Viscosity | Temp. T(X) |
| S11 | DVBA/ | HA1 | 3 | 22.47 | 402 |
| | VMXDA/ | | | | |
| | 3VPH | | | | |
| S12 | DVBA/ | HP4 | 3 | 24.75 | |
| | VMXDA/ | | | | |
| | 3VPH | | | | |
| C3 | DVBA/ | — | — | 21.7 | 381 |
| | VMXDA/ | | | | |
| | 3VPH | | | | |

Photo-cured layers were prepared from the photocurable compositions and analyzed via dynamic TGA the same way as described in Example 1. The TGA curves (not shown) were similar as for the samples of Example 1. The increase in the initial degradation temperature (T(X) from C3 to S11 was about 21° C.

### Example 4

### Investigation of Double Bond Conversion.

Fourier-transform infrared spectroscopy (FTIR) was conducted to measure the double bond conversion after a defined UV-curing regime of samples S1 to S8 (described in Example 1) and samples S11 and S12 (described in Example 3). For the measurements, the decrease of the peak for the double bond C═C in the IR spectrum at 1405 cm⁻¹ was measured before and after the curing, while the peak for the C—H benzene ring vibration at 713 cm−1 was used as internal reference. A Thermo Nicolet 6700 FTIR with DTGS TEC detector was used to record the FTIR spectra from 4000 cm⁻¹ to 625 cm⁻¹.

For the measurement a sample amount of 0.3 to 0.5 μl was dropped on a NaCl window (25 mm×25 mm) and thereafter covered with another NaCl window of the same size. The applied UV light intensity was 38 mW/cm² for a time of 132 seconds, which corresponds to a dosage of 5 J/cm2. The C═C conversion was calculated by the peak ratio of the cured sample (after exposure to the curing regime) to the uncured sample (at beginning of measurement, before UV exposure).

Table 7 provides a summary of the measured C═C conversion for the tested samples. It can be seen that when using VMXDA as polymerizable material, the addition of the hindered stabilizer had no negative influence on the conversion rate. When the combination of DVBA/VMXDA/3VPH was used as polymerizable material (samples S11, S12, C3), adding the hindered stabilizer HA1 (sample S11) caused an increase in the C═C conversion rate of 7.4%, and adding stabilizer HP4 (sample S12) resulted in an increase of 5.6% with respect to comparative sample C3.

TABLE 7

| | | Hindered | |
|---|---|---|---|
| Sample | Monomer | Stabilizer | C = C conversion [%] |
| S1 | VMXDA | HP1 - 1 part | 52.85 |
| S2 | VMXDA | HP2 - 1 part | 52.71 |
| S3 | VMXDA | HP3 - 1 part | 53.11 |
| S4 | VMXDA | HP4 - 1 part | 53.52 |
| S5 | VMXDA | HP4 - 3 parts | 53.68 |
| S6 | VMXDA | HA1 - 1 part | 52.35 |
| S7 | VMXDA | HA1 - 3 parts | 52.93 |
| C1 | VMXDA | — | 52.82 |
| S11 | DVBA/ | HA1 - 3 parts | 51.56 |
| | VMXDA/ | | |
| | 3VPH | | |
| S12 | DVBA/ | HP4 - 3 parts | 49.82 |
| | VMXDA/ | | |
| | 3VPH | | |
| C3 | DVBA/ | — | 44.15 |
| | VMXDA/ | | |
| | 3VPH | | |

### Example 5

### Shelf Life Investigation.

Shelf life tests were conducted to monitor the solution viscosity over time for curable compositions S11, S12, and C3 (see also description of the exact compositions in Example 3). The monitoring was conducted over the time period of 9 weeks, wherein the sample bottles were opened once a week to allow oxygen to contact the sample.

A summary of the results is shown in Table 8. It can be seen that the compositions containing the hindered amine stabilizer HA1 or hindered phenol stabilizer HP4 had after 9 weeks only very minor viscosity changes with a viscosity increase not greater than 0.6 mPa·s, while comparative composition C3, which did not include a hindered stabilizer of the present disclosure, had a viscosity increase of 1 mPa·s already after 6 weeks.

TABLE 8

| | | | Δ viscosity |
|---|---|---|---|
| Sample | Hindered Stabilizer | Time [weeks] | increase [mPa · s] |
| C3 | — | 9 | 1 |
| S11 | HA1 | 6 | 0.58 |
| S12 | HP4 | 6 | 0.35 |

### Carbon Content and Ohnishi Number

Further important properties for developing a suitable photocurable composition for IAP and NIL processing are the carbon content and Ohnishi number.

Table 9 shows a summary of the calculated percent carbon content and the Ohnishi number for the representative photocurable compositions described in Examples 1, 2, and 3.

The Ohnishi number (ON) is known to be an empirical parameter and was calculated as the ratio of total number of atoms (Nₜ) in the polymer repeat unit divided by the difference between the number of carbon atoms (Nc) and oxygen atoms (N_{O}) in the unit, ON=Nₜ/(N_{C}−N_{O}). For the calculation of the Ohnishi number, it was assumed that the cured materials contained 100 wt % of the polymerized monomer units formed by addition polymerization (no loss of atoms during polymerizations).

The carbon content was calculated as the percent of carbon atoms based on the total molecular weight of the compounds of the polymerizable material contained in the photocurable compositions.

It can be seen from Table 9, that photocurable compositions S1-S12 had a carbon content of 71 percent and greater, up to 93 percent, and Ohnishi numbers of 3.0 or lower.

TABLE 9

| Sample | Carbon Content [%] | Ohnishi Number |
|---|---|---|
| S1 | 71 | 3.0 |
| S2 | 71 | 3.0 |
| S3 | 71 | 3.0 |
| S4 | 71 | 3.0 |
| S5 | 71 | 3.0 |
| S6 | 71 | 3.0 |
| S7 | 71 | 3.0 |
| S8 | 71 | 3.0 |
| S9 | 93 | 1.9 |
| S10 | 93 | 1.9 |
| S11 | 80 | 2.5 |
| S12 | 80 | 2.5 |

### Viscosity Measurements

The viscosities were measured with a Brookfield DV-11+Pro viscometer using spindle #18. For each viscosity measurement, a sample of 6-7 ml was taken, added to the sample chamber and allowed to equilibrate for 15-20 minutes to reach the target temperature of 23° C. The viscosities were measured with spindle #18 at a speed of 135 rpm. For each sample, the measurement was three times repeated and an average value calculated.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any subcombination. Further, reference to values stated in ranges includes each and every value within that range. Many other embodiments may be apparent to skilled artisans only after reading this specification. Other embodiments may be used and derived from the disclosure, such that a structural substitution, logical substitution, or another change may be made without departing from the scope of the disclosure. Accordingly, the disclosure is to be regarded as illustrative rather than restrictive.

What is claimed is:

1. A photocurable composition comprising a polymerizable material, a hindered stabilizer, and a photoinitiator, wherein
the polymerizable material comprises at least one multi-functional aromatic vinyl monomer; and
the hindered stabilizer is a hindered amine of formula (1) or a hindered phenol of formula (2) 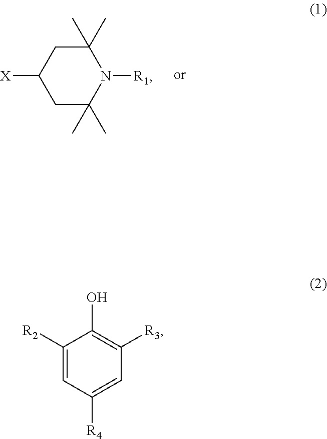
wherein X is H, CH₃, or Y—Z; Y is CH₂, O, S, or N; Z is an organic substituent;
R₁ is H, CH₃, OH, OR₅, CO—CH₃, or C(═O)R₅; R₂, R₃, R₄, R₅ is an organic substituent.

2. The photocurable composition of claim 1, wherein the hindered stabilizer comprises the hindered amine of formula (1), wherein X is Y—Z, with Z comprising at least one reactive C═C group.

3. The photocurable composition of claim 1, wherein the hindered stabilizer is a hindered amine selected from the group of:

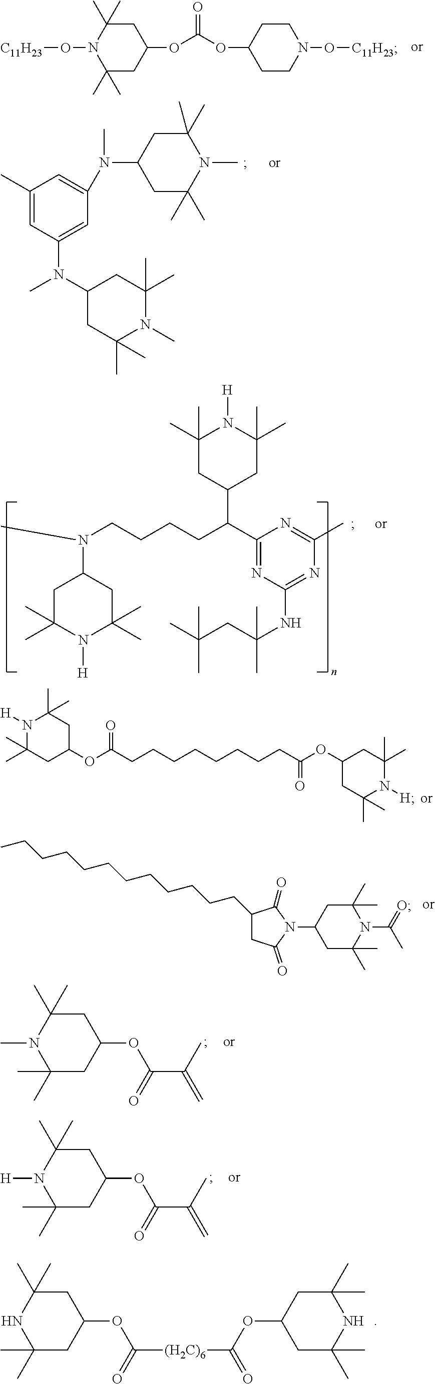

4. The photocurable composition of claim 1, wherein the hindered stabilizer has a molecular weight of at least 600 g/mol.

5. The photocurable composition of claim 1, wherein the hindered stabilizer is a hindered phenol selected from the group of:

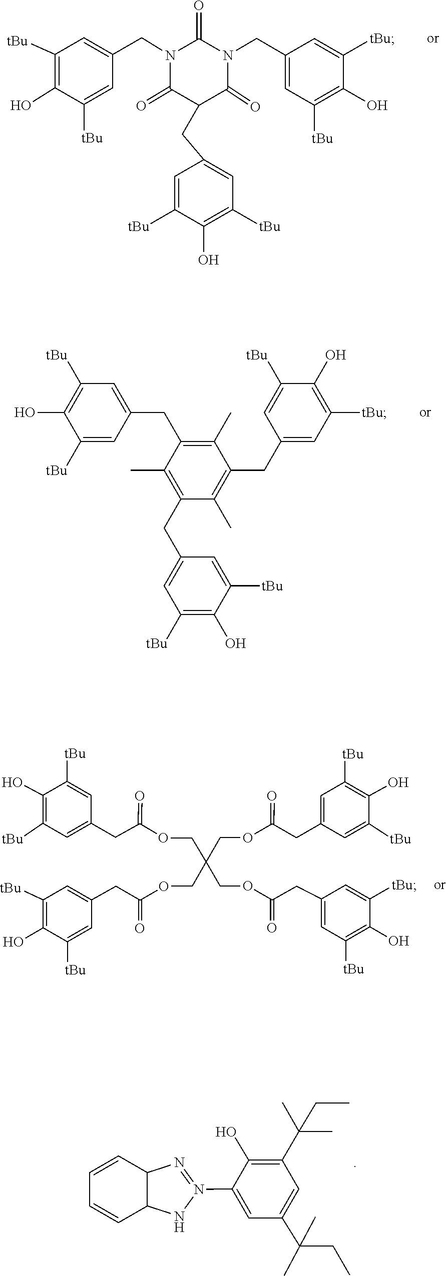

6. The photocurable composition of claim 1, wherein an amount of the hindered stabilizer is at least 0.5 wt % and not greater than 5 wt % based on the total weight of the photocurable composition.

7. The photocurable composition of claim 1, wherein the at least one multi-functional aromatic vinyl monomer includes a divinylbiphenyl monomer (DVBPh), or a trivinylbiphenyl monomer (TVBPh), or a trivinylphenyl monomer (TVPh), or a combination thereof.

8. The photocurable composition of claim 1, wherein the at least one multi-functional aromatic vinyl monomer comprises at least one vinyl group and at least one acrylate groups.

9. The photocurable composition of claim 8, wherein the multi-functional aromatic vinyl monomer is selected from:

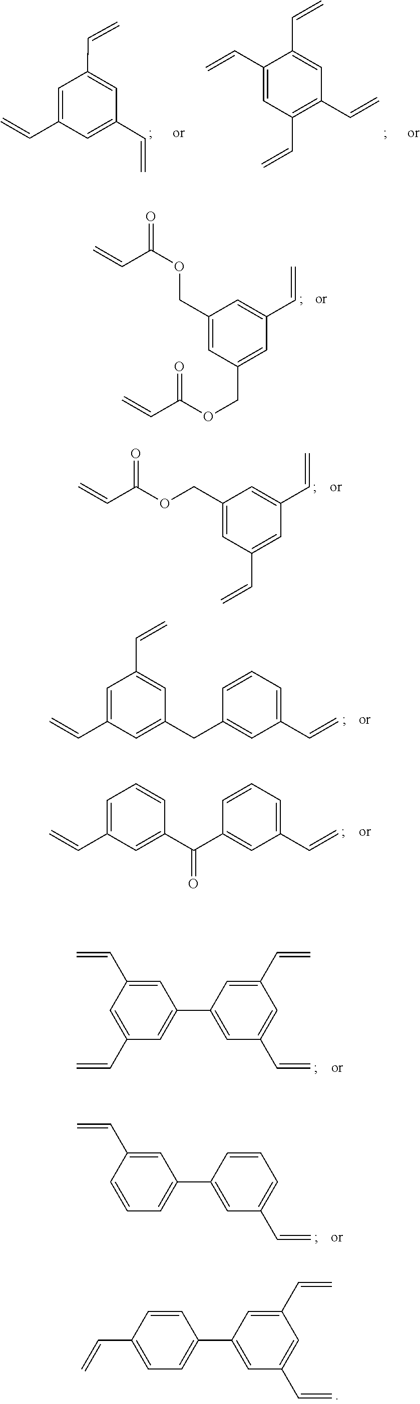

10. The photocurable composition of claim 1, wherein the amount of the multi-functional vinyl monomer is at least 80 wt % based on the total weight of the polymerizable material.

11. The photocurable composition of claim 10, wherein the amount of the multi-functional vinyl monomer is at least 95 wt % based on the total weight of the polymerizable material.

12. The photocurable composition of claim 1, wherein an amount of the polymerizable material is at least 90 wt % based on the total weight of the photocurable composition.

13. The photocurable composition of claim 1, wherein a carbon content of the photocurable composition after photo-curing is at least 71 percent.

14. The photocurable composition of claim 1, wherein a viscosity of the photocurable composition is not greater than 30 mPa s.

15. The photocurable composition of claim 1, wherein the photocurable composition is essentially free of a solvent.

16. A laminate comprising a substrate and a photo-cured layer overlying the substrate, wherein the photo-cured layer is formed from the photocurable composition of claim 1.

17. The laminate of claim 16, wherein an initial degradation temperature T(X) of the photo-cured layer is at least 330° C.

18. A method of forming a photo-cured layer on a substrate, comprising:
applying a layer of a photocurable composition on the substrate, wherein the photocurable composition comprises a polymerizable material, a hindered stabilizer, and a photoinitiator, wherein the polymerizable material comprises at least one multi-functional aromatic vinyl monomer; and the hindered stabilizer is a hindered amine of formula (1) or a hindered phenol of formula (2): 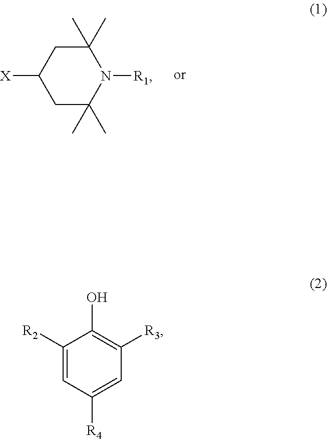
with X being H, CH₃, or Y—Z, wherein Y is CH₂, O, or S, or N, and Z is an organic substituent; R₁ is H, CH₃, OH, OR₅, or CO—CH₃ or COR₅; R₂, R₃, R₄, R₅ are organic substituents, the same or different;
bringing the photocurable composition into contact with a superstrate or an imprint template;
irradiating the photocurable composition with light to form a photo-cured layer; and
removing the superstrate or the imprint template from the photo-cured layer.

19. The method of claim 18, wherein an initial degradation temperature T(X) of the photo-cured layer is at least 330° C.

20. A method of manufacturing an article, comprising:
forming a photo-cured layer on a substrate according to claim 18;
forming a pattern on the substrate; and
processing the substrate on which the pattern has been formed in the forming; and
manufacturing the article from the substrate processed in the processing.
